(19) United States
(12) Patent Application Publication
(10) Pub. No.: US 2024/0257805 A1
(43) Pub. Date: Aug. 1, 2024

(54) AUTOMATIC SPEECH RECOGNITION SYSTEM CONTEXTUALLY BIASED FOR MEDICAL SPEECH

(71) Applicant: Verily Life Sciences LLC, South San Francisco, CA (US)

(72) Inventor: Joel Shor, Somerville, MA (US)

(21) Appl. No.: 18/423,490

(22) Filed: Jan. 26, 2024

Related U.S. Application Data
(60) Provisional application No. 63/482,522, filed on Jan. 31, 2023.

(51) Int. Cl. G10L 15/197; G10L 15/06; G10L 15/22; G16H 15/00

(52) U.S. Cl. CPC G10L 15/197; G10L 15/063; G10L 15/22; G16H 15/00

(57) ABSTRACT

Methods and systems of generating text representation of spoken medical speech are presented herein. Some methods may include the steps of providing a pre-trained automatic speech recognition (ASR) system stored in memory and executed on a processor; receiving, by the pre-trained ASR system, spoken medical speech; and generating text of the spoken medical speech by biasing the pre-trained ASR system using a contextual language model, where the contextual language model may include medical terminology that is not included in a vocabulary used to train the pre-trained ASR system.

### CROSS-REFERENCE TO RELATED APPLICATIONS

The present applications claims the benefit of and priority to, U.S. Provisional Patent Application No. 63/482,522, filed Jan. 31, 2023, the entirety of which is incorporated by reference herein.

### TECHNICAL FIELD

The present disclosure relates generally to contextually biasing automatic speech recognition systems to improve detection of medical speech.

### BACKGROUND

Advancements in artificial intelligence have led to a marked improvement in automatic speech recognition (ASR) systems and the increased adoption and reliance on voice interfaces in a variety of applications. Specifically, in the medical context, voice interfaces powered by robust ASR systems may be able to benefit physicians in a variety of ways. Voice is an easy way to control devices, especially when physicians have their hands busy. Moreover, voice interfaces may be able to recognize and transcribe speech during a medical appointment or procedure to produce a thorough report with little additional work by the physician.

However, developing ASR models that can accurately recognize medical speech is not straightforward. Standard ASR models often use training data that is collected outside of the medical context. For this reason, standard ASR models may not be capable of accurately identifying medical terms that are important and common in medical settings. Additionally, because ASR models require large amounts of speech to be adequately trained, collecting enough speech containing medical terminology from a variety of speakers can be challenging. Therefore, improved systems for and methods of developing ASR models that are able to accurately recognize medical speech are needed.

### SUMMARY

Methods of generating text representation of spoken medical speech are presented herein. Some methods may include the steps of providing a pre-trained automatic speech recognition (ASR) system stored in memory and executed on a processor; receiving, by the pre-trained ASR system, spoken medical speech; and generating text of the spoken medical speech by biasing the pre-trained ASR system using a contextual language model, where the contextual language model may include medical terminology that is not included in a vocabulary used to train the pre-trained ASR system.

In some embodiments, the pre-trained ASR system may include an acoustic model, a pronunciation model, and a language model that have been jointly trained using the vocabulary. In other embodiments, the pre-trained ASR system may include an acoustic model, a pronunciation model, and a language model that have been separately trained, and where the language model is trained using the vocabulary. In some embodiments, the biased ASR system may be a shallow fusion model. In some embodiments, the medical terminology may be a plurality of medical terms. In some embodiments, the contextual language model is a contextual n-gram language model. In some embodiments, the step of generating text of the spoken medical speech may include determining an n-gram score based on an overall model score generated by the pre-trained ASR system and a bias score generated by the contextual language model. In some embodiments, the language model may bias the ASR system during beam searching. In some embodiments, the language model may bias the ASR system before beam searching.

Some aspects of the present disclosure may include a method of generating a medical report. The method may include a method of generating text representation of spoken medical speech and writing a report based on the text of the medical speech.

A system for generating text of spoken medical speech is described herein. The system may include an input interface configured to receive spoken medical speech and a memory configured to store a plurality of processor-executable instruction. In some embodiments, the memory includes a pre-trained ASR system, a contextual language model, where the contextual language model may receive a plurality of medical terms, and a processor configured to execute the plurality of processor-executable instructions to perform operations. The operations may include biasing the pre-trained ASR system using the contextual language model and generating text of the spoken medical speech using the biased pre-trained ASR system, where at least one of the plurality of medical terms may not included in a vocabulary used to train the pre-trained ASR system.

In some embodiments, the biased pretrained ASR system may include an acoustic model, a pronunciation model, and a language model that have been jointly trained using the vocabulary. In some embodiments, the pre-trained ASR system may include an acoustic model, a pronunciation model, and a language model that have been separately trained, where the language model is trained using the vocabulary. In some embodiments, generating text of the spoken medical speech may include determining an n-gram score based on an overall model score generated by the pre-trained ASR system and a bias score generated by the contextual language model. In some embodiments, the contextual language model may bias the pre-trained ASR system during beam search decoding.

A non-transitory processor-readable storage medium storing a plurality of processor-executable instructions for generating text of spoken medical speech is described herein. In some embodiments, the instructions being executed by a processor to perform operations may include providing a pre-trained automatic speech recognition (ASR) model, biasing the pre-trained ASR model using a contextual language model, where the contextual language model may include medical terminology, and generating text of the spoken medical speech by biasing the pre-trained ASR system using a contextual language model, where the contextual language model comprises medical terminology that is not included in a vocabulary used to train the pre-trained ASR system.

In some embodiments, the pre-trained ASR system may include an acoustic model, a pronunciation model, and a language model that have been jointly trained using the vocabulary. In some embodiments, the pre-trained ASR system may include an acoustic model, a pronunciation model, and a language model that have been separately trained, and where the language model is trained using the vocabulary. In some embodiments, the contextual language model may be a contextual n-gram language model. In some embodiments, generating text of the spoken medical speech may include determining an n-gram score based on an overall model score generated by the pre-trained ASR system and a bias score generated by the contextual language model.

### BRIEF DESCRIPTION OF THE DRAWINGS

Illustrative embodiments of the present disclosure will be described with reference to the accompanying drawings, of which:

FIG. 1 is a schematic diagram illustrating a computer system 100 for implementing an ASR system 130, according to some embodiments of the present disclosure.

FIG. 2 is a block diagram 200 of an ASR system 130, according to some embodiments of the present disclosure.

FIG. 3 is a block diagram of a medical device 310 using an ASR system 130, according to some embodiments of the present disclosure.

FIG. 4 is a block diagram of a report generation system 400 using an ASR system 130, according to some embodiments of the present disclosure.

FIG. 5 is a flowchart of a method 500 of building and using an ASR system 130, according to some embodiments of the present disclosure.

FIG. 6 is a flowchart of a method of generating a textual representation 170 of spoken medical speech 150 using an ASR model 130 described herein, according to some embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, the term “network” may comprise any hardware or software-based framework that includes any artificial intelligence network or system, neural network or system and/or any training or learning models implemented thereon or therewith.

As used herein, the term “module” may comprise hardware or software-based framework that performs one or more functions. For example, a module may be implemented as one or more computer programs to be executed on programmable computers, each comprising some combination of at least one processor, a data storage system, at least one input device and/or at least one output device. In some embodiments, the module may be implemented on one or more neural networks.

A voice interface system may listen to or record words spoken in a natural language and may then process the audio to recognize and generate a textual representation of the spoken words. In the medical context, voice interface systems can be used in a variety of applications to make it easier for physicians to perform important tasks. For instance, voice interfaces may be incorporated into a medical device so that a physician may control the medical device with their voice, which may be particularly useful when the physician is using both hands. Moreover, voice interface systems may improve record keeping and diagnosis by recording medical appointments and procedures and generating more comprehensive reports without relying on a medical professional to take notes.

Voice interface systems may include an automatic speech recognition (ASR) system that analyzes the audio input to generate a textual representation of the words in the audio input. ASR systems may use artificial intelligence (AI), and in particular neural networks, to improve the accuracy of text generation. ASR systems may be trained on audio data including spoken words.

However, developing ASR systems that can accurately recognize and generate text of speech in a medical setting can be difficult. Medical speech is speech (e.g., a string of one or more words and/or phrases) that includes words and phrases that are rarely used outside of a medical setting. Thus, using standard ASR systems would be ineffective because they are trained on data from other sources, which are unlikely to be exposed to enough medical terminology to effectively recognize these terms. Moreover, because training an ASR system requires a lot of audio data, collecting enough audio data of medical speech to develop an accurate and useful ASR system may be challenging.

Therefore, there is a need for ASR systems that are able to accurately recognize medical terminology without requiring large amounts of additional training data from a medical setting. In some aspects, the present disclosure may describe various embodiments of ASR systems that use contextual biasing to tailor the system to recognize medical speech. In some embodiments, a standard, pre-trained ASR system may include an acoustic model, a pronunciation model, and a language model. The ASR system may further include a language model that receives medical terminology, where the medical terminology is rarely included or not included in the training data. The contextual language model may be used to bias the pre-trained ASR system to recognize medical terminology in the medical speech. In this way, an ASR system can be developed that may perform more accurate speech recognition of medical speech without additional training.

These descriptions are provided for example purposes only and should not be considered to limit the scope of the invention described herein. Certain features may be added, removed, or modified without departing from the spirit of the claimed subject matter.

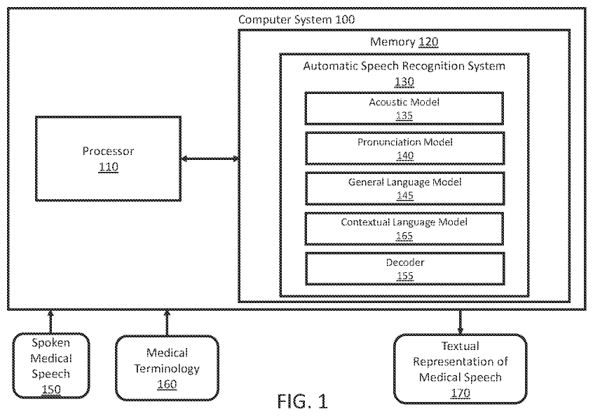

FIG. 1 is a schematic diagram illustrating a computer system 100 for implementing an ASR system 130, according to some embodiments of the present disclosure. The computer system 100 includes a processor 110 coupled to a memory 120. Although the computing device 100 is shown with only one processor 110, it is understood that processor 110 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in the computing device 100. The computing device 100 may be implemented as a stand-alone subsystem, as a board added to a computing device, and/or as a virtual machine.

The memory 120 may be used to store software executed by computing device 100 and/or one or more data structures used during operation of computing device 100. The memory 120 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor (e.g., the processor 110) or computer is adapted to read. In the present embodiments, for example, the memory 120 includes instructions suitable for training and/or using an ASR system 130 described herein.

The processor 110 and/or the memory 120 may be arranged in any suitable physical arrangement. In some embodiments, the processor 110 and/or the memory 120 are implemented on the same board, in the same package (e.g., system-in-package), on the same chip (e.g., system-on-chip), and/or the like. In some embodiments, the processor 110 and/or the memory 120 include distributed, virtualized, and/or containerized computing resources. Consistent with such embodiments, the processor 110 and/or the memory 120 may be located in one or more data centers and/or cloud computing facilities.

In some examples, the memory 120 may include non-transitory, tangible, machine readable media that includes executable code that when run by one or more processors (e.g., the processor 110) may cause the one or more processors to perform the methods described in further detail herein. For example, as shown, the memory 120 includes instructions for an ASR system 130, an acoustic model 135, a pronunciation model 140, a general language model 145, a contextual language model 165, and a decoder 155 that may be used to implement and/or emulate the systems and models, and/or to implement any of the methods described further herein. In some embodiments, the ASR system 130 may include one or more of an acoustic model 135, a pronunciation model 140, a general language model 145, a contextual language model 165, or a decoder 155. In some examples, the ASR model 130 may receive an input that includes spoken medical speech 150 and may generate a textual representation 170 of the spoken medical speech 150. In some embodiments, the contextual language model 165 may receive context-specific terminology, such as, for example, medical terminology 160. The contextual language model 165 may then bias the ASR model 130 to recognize the medical terminology 160.

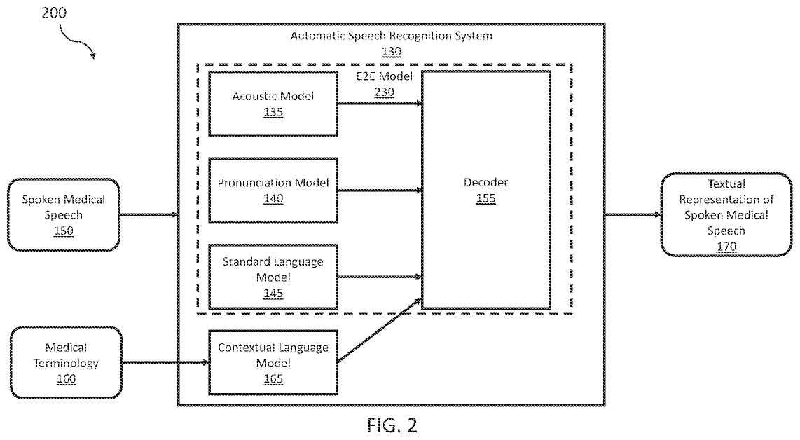

FIG. 2 is a block diagram 200 of an ASR system 130, according to some embodiments of the present disclosure. In this embodiment, the ASR system 130 includes an acoustic model 135, a pronunciation model 130 or lexicon, a general language model 145, a contextual language model 165, and a decoder 155. In some embodiments, the ASR system 130 may include any combination of an acoustic model 135, a pronunciation model 140, a general language model 145, a contextual language model 165, and a decoder 155. For example, in some embodiments, the ASR system 130 may include an acoustic model 135, a general language model 145, a contextual language model 165, and a decoder 155. In other embodiments, the ASR system 130 may include an acoustic model 135, a contextual language model 165, and a decoder 155.

The ASR system 130 may use AI and, in some embodiments, may include at least one neural network. In some embodiments, one or more of the acoustic model 135, pronunciation model 140, general language model 145, contextual language model 165, or decoder 155 may use AI and, in some embodiments, may include at least one neural network. In some embodiments, the ASR system 130 may include a pre-trained ASR model 230 in which one or more of the neural networks are trained with training data, as indicated by the dotted line in FIG. 2. The pre-trained ASR model 130 may not include the contextual language model 165, as described in more detail below.

In some embodiments, the pre-trained ASR system 230 may include a single neural network that includes an acoustic model 135, a pronunciation model 130, and/or a general language model 145, which may be referred to as an end-to-end (E2E) model. The E2E system may be trained such that the acoustic model 135, pronunciation model 140, and general language model 145 are trained simultaneously. In some embodiments, the E2E system may include a decoder 155 as a part of the single neural network or the decoder 155 may be separate. In other embodiments, the pre-trained ASR system 230 includes multiple neural networks. In some embodiments, each of the acoustic model 135, pronunciation model 140, and general language model 145 include a separate neural network, which may be referred to as a factor model. During training, each of the separate neural networks of the acoustic model 135, pronunciation model 140, and general language model 145 may be trained individually. In some embodiments, two or more of the separate neural networks of the acoustic model 135, pronunciation model 140, and general language model 145 may be trained simultaneously. In some embodiments, the decoder 155 may be trained with the acoustic model 135, pronunciation model 140, and general language model 145.

In some embodiments, the pre-trained ASR system 230 may be a standard ASR system trained with general speech. The speech may be collected from any appropriate sources in any appropriate way. For example, the speech may be audio recordings from a wide variety of speakers. The speech may be collected from any appropriate setting including, for example, a home, car, office, or any other appropriate setting or any combination of settings. In some embodiments, the training data may not include speech from a hospital, medical practice, nursing home, or any other medical setting. A vocabulary of the trained ASR system 130 may include words included in the training data. In some embodiments, one or more medical terms may not be included in the vocabulary.

Medical speech may be spoken word in natural language and may include medical terminology. Medical terminology may include medical terms or any words that are used in the practice of medicine or in discussion of biology. For example, medical terminology may include names of diseases, symptoms, anatomy, biology, biochemistry, procedures, billing codes, or any other appropriate medical terms. Medical speech may occur in any setting, but may particularly occur in a hospital, medical practice, nursing home, or any other medical setting.

The ASR system 130 may receive an input 150 of spoken medical speech 150 and may generate an output of a textual representation 170 of the spoken medical speech 150. The acoustic model 135 may receive the spoken medical speech 150 and may associate the raw acoustic features of that speech with phonetic or acoustic units. In some embodiments, the acoustic model 135 may also generate an acoustic score for each acoustic unit. The pronunciation model 140 may map the acoustic units to word or sub-word units based on pronunciations of various words. The general language model 145 may receive the word units from the acoustic model 135 and the pronunciation model 140 and assign a probability or score to each word unit. This probability or score may be referred to as the overall model score.

The ASR system 130 may also include a contextual language model 165 that may be contextually biased with medical terminology 160 to increase the likelihood of predicting the medical terminology 160 in the spoken medical speech 150. In some embodiments, a contextual language model 165 may be merged with or incorporated into the general language model 145. In some embodiments, the contextual language model 165 may be separate from the general language model 145 and, in some cases, may be external to the pre-trained ASR system 230 (which may be referred to as shallow fusion). The contextual language model 165 may be contextually biased in any other appropriate way. In some embodiments, the contextual language model 165 may include a contextual n-gram language model, which may be represented as a finite state transducer (FST).

The medical terminology 160 may be input in any appropriate form. In some embodiments, the medical terminology 160 may be a pre-built list of medical terms. In some embodiments, the pre-built list may be created by identifying commonly used medical terms from physicians and other medical professionals, from an analysis of medical documents, journal articles, or textbooks, or any other appropriate source. The medical terminology 160 may include text of medical terms. In some embodiments, the medical terminology 160 may include one or more recordings corresponding to one or more medical terms.

The contextual language model 165 may bias the overall model score produced by the pre-trained ASR system 230 to bias the ASR system 130 to recognize the medical terminology 160 in the spoken medical speech 150. The spoken medical speech 150 may contain a set of N acoustic observations x=(x₁, x₂, . . . , x_{N}) and there may be N word units y=(y₁, y₂, . . . , y_{N}) that correspond to the acoustic observations x. During beam search decoding, the contextual language model 165 may interpolate the score from the E2E model 230 according to the below equation:

$${y}^{*} = {arg max}_{y} log P ( y \vert x ) + λ log {P}_{C} ( y ) \qquad ( 1 )$$

where y* may represent an n-gram score for each word unit, P(y|x) may represent the overall model score generated by the pre-trained ASR system 230, and P_{C}(y) may represent the bias probability or score generated by the contextual language model 165. The term λ may be a tunable hyperparameter that controls how much the contextual language model 165 influences the overall model score during decoding. In some embodiments, the tunable hyperparameter may be changed to decrease the word error rate of the ASR system 130. Although equation (1) is discussed with respect to an E2E model, a person of ordinary skill would understand how to apply equation (1) to a factor model, such that a factor model can be used instead.

The decoder 155 receives the outputs of the acoustic model 135, pronunciation model 140, general language model 145, and the contextual language model 165 and generates phenome or word sequences. The decoder 155 may then generate a textual representation 170 of the spoken medical speech 150. The textual representation 170 may include a medical term or word.

The decoder 155 may include a beam search decoder. The contextual language model 165 may be applied during beam searching to bias the decoder 155 to include the medical terminology in the beam if applicable. In some embodiments, the contextual language model 165 may be applied before beam pruning. However, in some embodiments, the contextual language model 165 may be applied after beam pruning in some embodiments. In some embodiments, the decoder 155 receives an overall model score from the pre-trained ASR 230 and a bias score from the contextual language model 165. The beam search decoder may determine an n-gram score based on the overall model score and the bias score according to Equation 1. The n-gram score may then be used to prune the beam accordingly such that the decoder 155 is biased to recognize the medical terminology when forming word sequences, if applicable.

The contextual language model 165 may be beneficial because it allows a general pre-trained ASR system 230 to be used in a medical context without additional training. The contextual language model 165 may receive a list of medical terminology 160 and can then bias the ASR system 130 to recognize the medical terminology 160 even if there are no medical terms in the vocabulary of the pre-trained ASR system 130. Thus, the contextual language model 165 may decrease the word error rate in the medical setting without the onerous process of collecting large amounts of medical speech and training the ASR system 230 on the medical speech.

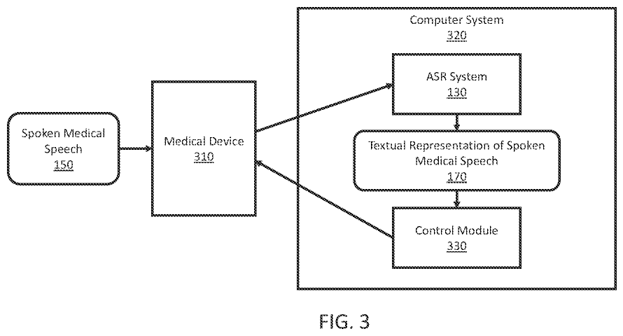

FIG. 3 is a block diagram of a medical device 310 using an ASR system 130, according to some embodiments of the present disclosure. In some embodiments, a medical device 310 may be an endoscope, a catheter, robotic surgical tools, imaging devices using ultrasound or magnetic resonance imaging (MRI), smart hospital beds, automatic scribes or other note-taking tools, or may be any other appropriate device for use in a medical setting. The medical device 310 may receive spoken medical speech 150. In some embodiments, the medical device 310 may include a microphone or other component for receiving spoken words. The medical device 310 may then pass the medical speech 150 to a computer system 320. In some embodiments, the computer system 320 may be located within the medical device 310, but in other embodiments, the computer system 320 may be located external to, but in communication with, the medical device 310. The computer system 320 may include an ASR system 130 according to any embodiment described herein. The computer system 320 may further include a control module 330. The ASR system 130 may receive the medical speech 150 and generate a textual representation 170 of the medical speech 150. The ASR system 130 may pass the textual representation 170 along to the control module 330. The control module 330 may analyze the meaning of the textual representation 170 to determine a meaning of the spoken medical speech 150. The control module 330 may then control the medical device 310 in response to the meaning of the spoken medical speech 150. For example, control module 330 may be implemented as a set of instructions in software on the computer system 320, which may be programmable, for receiving a textual representation 170 of the medical speech 150 and analyzing the textual representation 170 to understand an instruction and implement the instruction using the medical device 310.

In one example, the medical device 310 may be an endoscope, which a physician may use for colonoscopies. During a colonoscopy procedure, the physician may speak aloud with instructions to move the camera of the endoscope such as “move the camera to the right” or “move the camera up”. The endoscope may receive the instructions and pass them along to the computer system 320. The computer system 320 may use the ASR system 130 to generate a textual representation of the instructions and use the control module 330 to control the endoscope according to the instructions. In another example, the medical device 310 may be an MRI system. The medical professional operating the MRI system may say “start MRI scan” or “increase brightness”. The MRI system may receive the instructions and pass them along to the computer system 320. The computer system may use the ASR system 130 to generate a textual representation of the instructions and use control module 330 to control the MRI system according to the instructions.

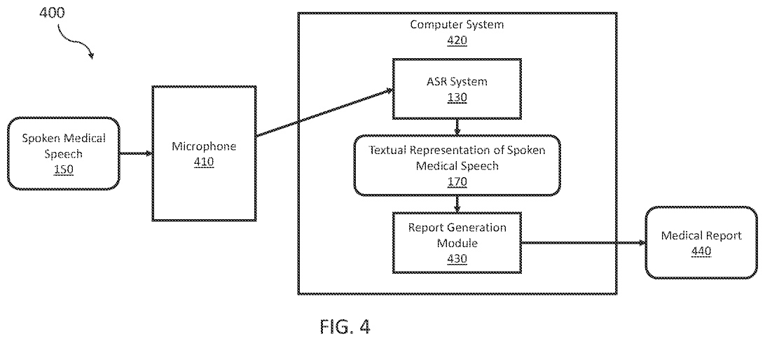

FIG. 4 is a block diagram of a report generation system 400 using an ASR system 130, according to some embodiments of the present disclosure. In some embodiments, a microphone 410 or other device may receive spoken medical speech 130. The microphone 410 may then pass the medical speech 150 to a computer system 420. In some embodiments, the computer system 420 may be located external to, but in communication with, the microphone 410. The computer system 420 may include an ASR system 130 according to any embodiment described herein. The computer system 420 may further include a report generation module 430, which may generate a report 440 based on the spoken medical speech 150. For example, the report generation module 430 may be implemented as a set of instructions in software on the computer system 420, which may be programmable, for receiving a textual representation 170 of the medical speech 150 and formatting the textual representation 170 into a report according to the desired application. The report generation module 430 may collect a series of words or textual representations 170 over some time period, representative of a number of words and/or phrases, and automatically generate a medical report 440. The ASR system 130 may receive the medical speech 150 and generate a textual representation 170 of the medical speech 150. The ASR system 130 may pass the textual representation 170 along to the report generation module 430. The report generation module 430 may analyze the meaning of the textual representation 170 to determine a meaning of the spoken medical speech 150. The report generation module 430 may then generate a report 440 based on the meaning of the spoken medical speech 150.

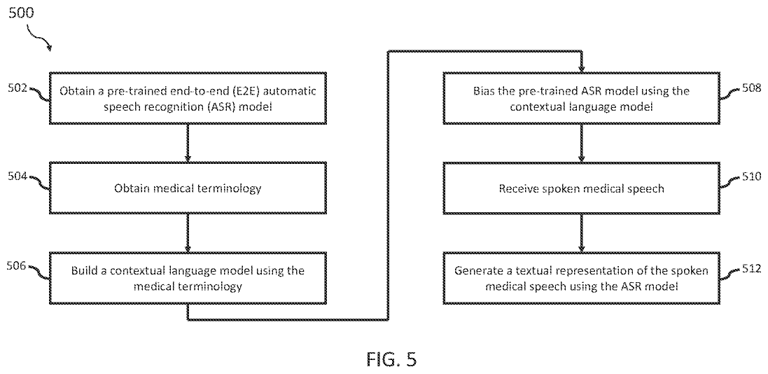

FIG. 5 is a flowchart of a method 500 of building and using an ASR system 130, according to some embodiments of the present disclosure. The steps of the method 500 may be implemented, at least in part, in the form of executable code stored on non-transitory, tangible, machine-readable media that, when run by one or more processors may cause the one or more processors to perform one or more of the steps of the method 500.

Step 502 may include obtaining a pre-trained ASR system 230, which in some cases may be an E2E model. In some cases, the E2E model may be a LAS model. The pre-trained ASR system 230 may be a general ASR system that is trained on spoken words in natural language. The training data may be collected from one or more settings including homes, cars, offices, or any other appropriate setting. In some embodiments, the training data may not be collected from medical settings including, for example, hospitals, medical practices, nursing homes, or any other appropriate medical setting. In some embodiments, the vocabulary of the pre-trained ASR system 230 may not include one or more medical terms in the medical terminology.

Step 504 may include obtaining medical terminology 160. The medical terminology 160 may include medical terms that are out of vocabulary (OOV) of the pre-trained ASR system 230. In some embodiments, some of the medical terms in the medical terminology 160 are in the vocabulary of the pre-trained ASR system 230. In some embodiments, the medical terminology may include names of diseases, symptoms, anatomy, biology, biochemistry, procedures, billing codes, or any other appropriate medical terms. The medical terminology 160 may be collected in any appropriate way. In some embodiments, the medical terminology 160 may be collected from physicians or other medical professionals. In some embodiments, the medical terminology 160 may be collected from medical journal articles, documents, or textbooks.

Step 506 may include building a contextual language model 165 using the medical terminology 160. The contextual language model 165 may be a contextual n-gram language model, which may be represented as a finite state transducer (FST). The medical terminology 160 may be input into the contextual language model 165.

Step 508 may include biasing the pre-trained ASR system 230 using the contextual language model 165. The contextual language model 165 may be external to the pre-trained ASR system 230. When using an E2E model, the contextual language model 165 may not be a part of the same neural network as the E2E model. The contextual language model 165 may bias the ASR system 130 during beam searching. In some embodiments, the biasing occurs before beam searching. In some embodiments, the contextual language model 165 biases the ASR system 130 according to Equation 1, as described herein.

Step 510 may include receiving spoken medical speech 150. Spoken medical speech 150 may be input into the ASR system 130. The spoken medical speech 150 may include medical terminology.

Step 512 may include generating a textual representation 170 of the spoken medical speech 150 using the ASR model 130. The contextual language model 165 may bias the pre-trained ASR system 230 to recognize medical terminology in the spoken medical speech 150. The decoder 155 may use the output of the pre-trained ASR model 230 and the contextual language model 165 to generate a textual representation 170 of the spoken medical speech 150.

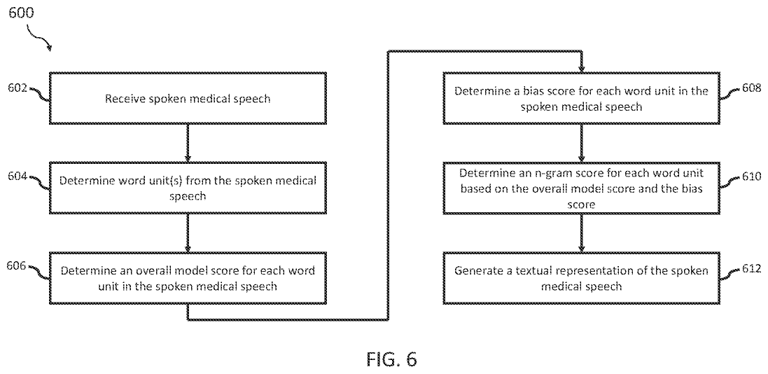

FIG. 6 is a flowchart of a method of generating a textual representation 170 of spoken medical speech 150 using an ASR model 130 described herein, according to some embodiments of the present disclosure. The steps of the method 600 may be implemented, at least in part, in the form of executable code stored on non-transitory, tangible, machine-readable media that, when run by one or more processors may cause the one or more processors to perform one or more of the steps of the method 600.

Step 602 may include receiving spoken medical speech 150. Spoken medical speech 150 may be input into the ASR system 130. The spoken medical speech 150 may include medical terminology. The medical terminology may include medical terms that are out of vocabulary (OOV) of the pre-trained ASR system 230. The medical speech 150 may also include words or phrases that are in the vocabulary of the pre-trained ASR system 230. In some embodiments, some of the medical terms in the medical terminology are in the vocabulary of the pre-trained ASR system 230. In some embodiments, the medical terminology may include names of diseases, symptoms, anatomy, biology, biochemistry, procedures, billing codes, or any other appropriate medical terms.

Step 604 may include determining one or more word units from the spoken medical speech 150. An ASR system 130 may include an acoustic model 135, which may receive acoustic occurrences of the spoken medical speech and generate acoustic units. The ASR system 130 may also include a pronunciation model 140, which may generate word units from the acoustic units generated by the acoustic model 135.

Step 606 may include determining an overall model score for each word unit in the spoken medical speech 150. The ASR system 130 may further include a general language model 145. The general language model 145 may generate a probability or overall model score for each word unit generated by the acoustic model 135 and pronunciation model 140.

Step 608 may include determining a bias score for each word unit in the spoken medical speech 150. The ASR system 130 may also include a contextual language model 165. The contextual language model 165 may be separate from the acoustic model 135, pronunciation model 140, and the general language model 145. The contextual language model 165 may receive an input of medical terminology 160. The contextual language model 165 may generate a bias score for the word units based on the medical terminology 160.

Step 610 may include determining an n-gram score for each word unit based on the overall model score and the bias score. The contextual language model 165 may bias the ASR system 130 such that the decoder 155 will assign a higher probability to medical terms in the medical terminology when decoding the word units into words. The n-gram score may be calculated using Equation 1 herein. The n-gram score may be applied during beam forming and, in some embodiments, before beam pruning.

Step 612 may include generating a textual representation 170 of the spoken medical speech 150. The contextual language model 165 may bias the pre-trained ASR system 230 to recognize medical terminology in the spoken medical speech 150. The decoder 155 may use the output of the pre-trained ASR model 230 and the contextual language model 165 to generate a textual representation 170 of the spoken medical speech 150.

A number of variations are possible on the examples and embodiments described above. Accordingly, the logical operations making up the embodiments of the technology described herein are referred to variously as operations, steps, objects, elements, components, layers, modules, or otherwise. Furthermore, it should be understood that these may occur in any order, unless explicitly claimed otherwise or a specific order is inherently necessitated by the claim language.

Generally, any creation, storage, processing, and/or exchange of user data associated with the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In some embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

In several example embodiments, the elements and teachings of the various illustrative example embodiments may be combined in whole or in part in some or all of the illustrative example embodiments. In addition, one or more of the elements and teachings of the various illustrative example embodiments may be omitted, at least in part, and/or combined, at least in part, with one or more of the other elements and teachings of the various illustrative embodiments.

Any spatial references such as, for example, “upper,” “lower,” “above,” “below,” “between,” “bottom,” “vertical,” “horizontal,” “angular,” “upwards,” “downwards,” “side-to-side,” “left-to-right,” “right-to-left,” “top-to-bottom,” “bottom-to-top,” “top,” “bottom,” “bottom-up,” “top-down,” etc., are for the purpose of illustration only and do not limit the specific orientation or location of the structure described above. Connection references, such as “attached,” “coupled,” “connected,” and “joined” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

Additionally, the phrase “at least one of A and B” should be understood to mean “A, B, or both A and B.” The phrase “one or more of the following: A, B, and C” should be understood to mean “A, B, C, A and B, B and C, A and C, or all three of A, B, and C.” The phrase “one or more of A, B, and C” should be understood to mean “A, B, C, A and B, B and C, A and C, or all three of A, B, and C.”

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims.

What is claimed is:

1. A method of generating text of medical speech, the method comprising:
providing a pre-trained automatic speech recognition (ASR) system stored in memory and executed on a processor;
receiving, by the pre-trained ASR system, spoken medical speech; and
generating text of the spoken medical speech by biasing the pre-trained ASR system using a contextual language model, wherein the contextual language model comprises medical terminology that is not included in a vocabulary used to train the pre-trained ASR system.

2. The method of claim 1, wherein the pre-trained ASR system comprises an acoustic model, a pronunciation model, and a language model that have been jointly trained using the vocabulary.

3. The method of claim 1, wherein the pre-trained ASR system comprises an acoustic model, a pronunciation model, and a language model that have been separately trained, and wherein the language model is trained using the vocabulary.

4. The method of claim 1, wherein the biased ASR system is a shallow fusion model.

5. The method of claim 1, wherein the medical terminology comprises a plurality of medical terms.

6. The method of claim 1, wherein the contextual language model is a contextual n-gram language model.

7. The method of claim 6, wherein the step of generating text of the spoken medical speech comprises determining an n-gram score based on an overall model score generated by the pre-trained ASR system and a bias score generated by the contextual language model to generate a textual representation of a medical term that is not included in the vocabulary used to train the pre-trained ASR system.

8. The method of claim 1, wherein the language model biases the ASR system during beam searching.

9. The method of claim 1, wherein the language model biases the ASR system before beam searching.

10. A method of generating a medical report, comprising:
the method of claim 1; and,
writing a report based on the text of the medical speech.

11. A system for generating text of spoken medical speech comprising:
an input interface configured to receive spoken medical speech;
a memory configured to store a plurality of processor-executable instruction, the memory including:
a pre-trained ASR system; and,
a contextual language model, wherein the contextual language model receives a plurality of medical terms; and,
a processor configured to execute the plurality of processor-executable instructions to perform operations including:
biasing the pre-trained ASR system using the contextual language model; and,
generating text of the spoken medical speech using the biased pre-trained ASR system, wherein at least one of the plurality of medical terms is not included in a vocabulary used to train the pre-trained ASR system.

12. The system of claim 11, wherein the biased pretrained ASR system comprises an acoustic model, a pronunciation model, and a language model that have been jointly trained using the vocabulary.

13. The system of claim 11, wherein the pre-trained ASR system comprises an acoustic model, a pronunciation model, and a language model that have been separately trained, and wherein the language model is trained using the vocabulary.

14. The system of claim 12, wherein generating text of the spoken medical speech comprises determining an n-gram score based on an overall model score generated by the pre-trained ASR system and a bias score generated by the contextual language model.

15. The system of claim 11, wherein the contextual language model biases the pre-trained ASR system during beam search decoding.

16. A non-transitory processor-readable storage medium storing a plurality of processor-executable instructions for generating text of spoken medical speech, the instructions being executed by a processor to perform operations comprising:
providing a pre-trained automatic speech recognition (ASR) model;
biasing the pre-trained ASR model using a contextual language model, wherein the contextual language model comprises medical terminology; and,
generating text of the spoken medical speech by biasing the pre-trained ASR system using a contextual language model, wherein the contextual language model comprises medical terminology that is not included in a vocabulary used to train the pre-trained ASR system

17. The non-transitory processor-readable storage medium of claim 16, wherein the pre-trained ASR system comprises an acoustic model, a pronunciation model, and a language model that have been jointly trained using the vocabulary.

18. The non-transitory processor-readable storage medium of claim 16, wherein the pre-trained ASR system comprises an acoustic model, a pronunciation model, and a language model that have been separately trained, and wherein the language model is trained using the vocabulary.

19. The non-transitory processor-readable storage medium of claim 16, wherein the contextual language model is a contextual n-gram language model.

20. The non-transitory processor-readable storage medium of claim 19, wherein generating text of the spoken medical speech comprises determining an n-gram score based on an overall model score generated by the pre-trained ASR system and a bias score generated by the contextual language model.
